(19) United States
(12) Patent Application Publication
(10) Pub. No.: US 2005/0238581 A1
(43) Pub. Date: Oct. 27, 2005

(54) Dynamic hepatic recycling glucose tolerance test

(76) Inventors: Irwin J. Kurland, Lloyd Harbor, NY (US); W. N. Paul Lee, Palos Verdes Estates, CA (US); Mohammed Saad, Pasadena, CA (US); Jun Xu, Diamond Bar, CA (US)

Correspondence Address: JOHN P. O'BANION;O'BANION & RITCHEY LLP, 400 CAPITOL MALL SUITE 1550, SACRAMENTO, CA, 95814, US

(21) Appl. No.: 11/060,640

(22) Filed: Feb. 16, 2005

Related U.S. Application Data
(63) Continuation of application No. PCT/US03/25606, filed on Aug. 16, 2003.
(60) Provisional application No. 60/404,255, filed on Aug. 16, 2002.

(51) Int. Cl. A61K 49/00; C12Q 1/54

(52) U.S. Cl. 424/9.2; 435/14

(57) ABSTRACT

Systems and methods are described providing a hepatic recycling glucose tolerance test for the diagnosis of types and subtypes of diabetes mellitus and other hyperglycemic or hypoglycemic conditions. A method is also provided for screening candidate drugs for treating various types of abnormal glucose metabolism and to monitor whether the course of treatment is effective. The method also allows the correlation of gene activity, hormone and metabolite levels with glucose flux and recycling and an assessment of the degree of hepatic insulin resistance. The method utilizes a preferably non-radioactive stable labeled glucose to asses the relative rates of carbon flow in the liver and provides a hepatic recycling constant that is a measure of the relative rate of glucose recycling. The labeled glucose may be introduced to the patient orally, intravenously or by intraperitoneal administration for the desired effect.

### CROSS-REFERENCE TO RELATED APPLICATIONS

This application claims priority from, and is a 35 U.S.C. § 111 (a) continuation of, co-pending PCT international application serial number PCT/US2003/025606 filed on Aug. 16, 2003 and which designates the U.S., incorporated herein by reference in its entirety, which claims priority from U.S. provisional application Ser. No. 60/404,255 filed on Aug. 16, 2002, incorporated herein by reference in its entirety.

### STATEMENT REGARDING FEDERALLY SPONSORED RESEARCH OR DEVELOPMENT

This invention was made with Government support under Grant Nos. CA42710, DK56090, DK58132, and RR00425 awarded by the National Institutes of Health. The Government has certain rights in this invention.

### INCORPORATION-BY-REFERENCE OF MATERIAL SUBMITTED ON A COMPACT DISC

Not Applicable

### BACKGROUND OF THE INVENTION

1. Field of the Invention

This invention pertains generally to diagnostic testing protocols for identifying and treating physiological and pathophysiological conditions in mammals in the laboratory and in humans in the clinic, and more particularly to diagnostic tests to screen putative pharmacological agents for the treatment of hyperglycemia, identification of gene activity associated with hyperglycemia, and the identification of peripheral versus hepatic insulin sensitivity.

2. Description of Related Art

Chronic hyperglycemia has been shown to cause damage to the eyes, kidneys, nerves, heart and blood vessels and is a significant medical condition affecting a substantial percentage of the population of the world. Hyperglycemia resulting from diabetes mellitus can be a major medical problem with high morbidity and mortality.

Diabetes mellitus is a condition in which high blood glucose can result from a number of enzymatic and metabolic disorders involving the muscle, fat, islet cells, and the liver. The American Diabetes Association (ADA) classifies diabetes mellitus into two types. The first type, Type 1 diabetes, typically appears at a young age and is characterized by clearly deficient insulin production. The second and more common type of diabetes is Type 2 diabetes, which is seen most frequently among obese older adults and is characterized by insulin resistance as well as a slightly decreased insulin secretion.

Research over the past twenty years has increased the general understanding of the molecular mechanisms contributing to the development of hyperglycemia and associated secondary conditions in patients. Much of this research used animal models to determine the basic mechanisms of glucose utilization that could then be applied to evaluate human diseases.

Generally, the baseline production of endogenous glucose in the body is normally balanced with the tissue utilization of glucose. Approximately 85% of endogenous glucose production occurs in the liver and the remaining production by the kidneys. Typically about half of baseline hepatic glucose production is obtained from glycogenolysis and half from gluconeogenesis.

The balance between endogenous glucose production and tissue glucose uptake is upset following the ingestion of glucose producing an increase in plasma glucose levels. An increase in the concentration of glucose in plasma stimulates the release of insulin from the pancreatic beta cells producing a temporary state of hyperinsulinemia and hyperglycemia in plasma.

The combined effects of increased insulin levels and hyperglycemia is to stimulate three tightly coupled mechanisms: (a) the suppression of endogenous glucose production primarily in the liver; (b) the stimulation of glucose uptake by the liver and gastrointestinal tissues, and (c) the stimulation of glucose uptake by peripheral tissues, primarily muscle. Therefore, the maintenance of plasma glucose homeostasis depends upon a normal insulin secretory response by the pancreatic beta cells as well as normal tissue sensitivity to insulin and hyperglycemia to modulate glucose utilization.

Poor insulin production in Type 1 diabetes, for example, leads to insufficient concentrations of insulin in plasma to influence the metabolic system. Insulin resistance and normal glucose tolerance, on the other hand, characterize type 2 diabetes, in the early stages of the disease. Over time the body increases insulin production to compensate that can lead to impaired glucose tolerance. Eventually, the defective beta cells become depleted, further contributing to the cycle of glucose intolerance and hyperglycemia.

Because the etiology and pathophysiology among patients with diabetes mellitus can be markedly different, the use of a variety of different treatments, screening methods and prevention strategies are required. In addition, continuing medical investigation into the contribution of genetic and physiological causative factors are essential for the development of new drug compositions and treatments for hyperglycemia.

Current diagnostic tests for diabetes include the random plasma glucose, fasting plasma glucose, glycosylated hemoglobin (HbA_{1c}) measurements, and oral glucose-tolerance tests. The glucose tolerance test is currently the principal test for the diagnosis of glucose intolerance and early diabetes. The oral glucose tolerance test typically consists of drinking a 100 g glucose solution and measuring the blood glucose (bG) values at selected time points to produce a curve. The blood glucose excursion after a glucose load is used to characterize glucose intolerance due to insulin deficiency or resistance. Since the observed plasma glucose and insulin responses during the oral glucose tolerance test reflect the ability of pancreatic beta-cells to secrete insulin and the sensitivity of other tissues to insulin, the glucose tolerance test can be used as an indicator of beta-cell function and insulin resistance.

Because blood glucose concentration after a glucose load is the result of the balance between glucose uptake and glucose release, previous studies have examined the role of hepatic clearance of absorbed glucose or suppression of endogenous production as the mechanism for glucose intolerance in diabetes. Since hepatic glucose uptake and release share the same metabolic network of enzymes and intermediates, it has been observed that extensive glucose recycling occurs during a glucose tolerance test. The fasting glucose level may be elevated in Type 2 diabetes due to hepatic insulin resistance, defined as the resistance to insulin's action in the liver to restrain glucose production as well as the excessive recycling of glucose carbon (termed flux) during an overnight fast. Elevated post-prandial glucose excursions may also result, in part, from resistance to insulin's action to speed glucose transport into the periphery (muscle and fat tissues). Consequently, conventional glucose tolerance tests cannot distinguish the contribution of pathophysiology at the level of the liver versus the periphery in the development of hyperglycemia associated with Type 2 diabetes.

Accordingly, there is a need for a test that can differentiate between hepatic and peripheral insulin sensitivity and that will provide a diagnostic test for diabetes and other conditions producing hyperglycemia. There is also a need for a method for correlating insulin action with the activity of genes thought to be associated with diabetes. The present invention provides for these needs, as well as others, and generally overcomes the deficiencies found in the background art.

### BRIEF SUMMARY OF THE INVENTION

It is generally accepted that an abnormal response to a standard glucose challenge in the form of a glucose tolerance test is an indication of clinical diabetes. The degree of blood glucose elevation and the rapidity that it is cleared from plasma during a traditional glucose tolerance test constitute the criteria for separating patients into normal, glucose intolerant and diabetes groups. Since blood glucose concentration after a glucose load is the balance between glucose uptake and glucose release, previous research has examined the role of hepatic clearance of absorbed glucose or suppression of endogenous production as the mechanism for glucose intolerance in diabetes.

According to one aspect of the invention, a hepatic recycling constant, (k_{HR}), is derived which has the potential to be a major tool for testing the hepatic action of drugs used to treat both Type 1 and Type 2 diabetes mellitus.

Another aspect of the invention provides a method for evaluating the pharmacogenetic profile of patients to be treated with an anti-diabetic drug. Type 2 diabetes is known to have many subtypes that are a function of whether the primary metabolic defect is centered on a dysfunction of insulin action in muscle, liver, adipose tissue, or if the result is due to a dysfunction in pancreatic insulin secretion. It is also known that a dysfunction in insulin action in one tissue or organ can result in a secondary disturbance in insulin action in another tissue or organ. The hepatic recycling constant, (k_{HR}), is indicative of hepatic insulin and glucose action, and this can be used to evaluate whether the primary effect of a drug, or the primary site of dysregulation in a subtype of Type 2 diabetes mellitus, involves the liver.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWINGS

The invention will be more fully understood by reference to the following drawings which are for illustrative purposes only:

FIG. 1 is a block diagram of one embodiment of the invention adapted for screening of candidate drugs for hyperglycemia treatment.

FIG. 2A is a graph depicting the time course of the appearance of M0, M1 and M2 glucose isotopomers according to one aspect of the present invention.

FIG. 2B is a graph depicting the time course of the generation of M1 glucose isotopomer and the ratio of plasma M1 to M2 isotopomer.

FIG. 3 is a graph of the plasma insulin concentration according to one aspect of the invention.

FIG. 4A is a graph of the time course of M2 lactate isotopomer according to one aspect of the invention.

FIG. 4B is a graph of the time course of mean lactate concentration according to one aspect of the invention.

FIG. 4C is a graph of the time course of PC flux according to one aspect of the invention.

FIG. 5A is a graph of the time course of M1 isotopomer produced as a fraction of the labeled glucose pool according to one aspect of the invention.

FIG. 5B is a graph of the time course of M2 isotopomer produced as a fraction of the labeled glucose pool according to one aspect of the invention.

FIG. 5C is a graph of the time course of the M1/M2 ratio of plasma glucose isotopomers according to one aspect of the present invention.

FIG. 6 is a bar graph of the ratio of labeled carbon.

FIG. 7A-7C are western blot results showing the time course of glucokinase, G6PDH and PEPCK expression respectively.

FIG. 8A-8D are graphs of the time course of the change in total glucose, M0 glucose isotopomer, M1 glucose isotopomer and M2 isotopomer respectively for C57BL/6 and PPARα KO mice according to the present invention.

FIG. 9 is a graph of the time course of the M1/M2 ratio of plasma glucose isotopomers for C57BL/6 and PPARα KO mice according to one aspect of the present invention.

FIG. 10 is a graph of the time course of the percent difference between the plasma [2-²H]- and [6, 6-²H₂]-glucose enrichments during an alternative glucose tolerance test according to the present invention.

### DETAILED DESCRIPTION OF THE INVENTION

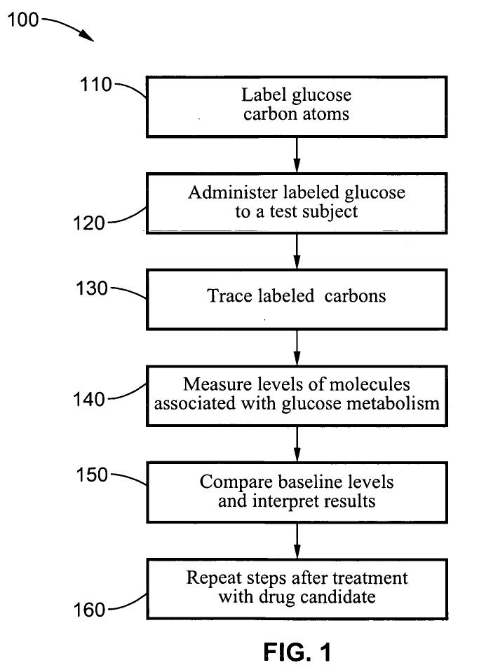

Referring more specifically to the drawings, for illustrative purposes the present invention is embodied in the methods and apparatus generally shown in FIG. 1 through FIG. 10. It will be appreciated that the methods may vary as to the specific steps and sequence, without departing from the basic concepts as disclosed herein.

The present invention provides substantial advancement beyond past diagnostic research and investigation studying the disposition of glucose molecules through metabolic pathways in diseased and normal individuals, and provides diagnostic applications of these methods to generally observed hyperglycemic or hypoglycemic conditions, screening for new drug candidates and to the investigation of normal and abnormal gene activity.

Referring first to FIG. 1, one embodiment of the method 100 adapted for evaluating the function of the glucose utilization system of the body of a test subject as well as screening new drugs for treatment of hyperglycemic conditions is shown in a block diagram for illustration. It will be seen that the hepatic recycling glucose tolerance test of the invention assesses the relative rates of glucose carbon flow (termed flux) in and out of liver cells during a glucose tolerance test. The method 100 estimates a hepatic recycling constant (k_{HR}), that is a measure of the relative rate of re-circulating of glucose through hepatic glucokinase and glucose-6-phosphatase. Since glucose-6-phosphate (the predominant form of glucose in the liver cells) is extensively exchanged with glycogen, glucose and intermediates of the hepatic pentose phosphate pathways, the hepatic recycling constant, (k_{HR}), reflects the substrate fluxes through these pathways and liver insulin sensitivity.

Liver insulin sensitivity determines the production of glucose by the liver in the fasting state, as well as the amount of glycogen and the net hepatic glucose output during and after meals. Resistance to the hepatic action of insulin is the major factor governing the fasting plasma glucose concentration, and contributes to post-prandial excursion in the plasma glucose level. The estimation of (k_{HR}) provides a new method for assessing the degree of hepatic insulin resistance seen in the various sub-types of adult-onset, Type 2 diabetes mellitus, as well as provides an assessment for the hepatic action of anti-diabetic drugs.

At block 110, preferably two carbon atoms of glucose molecules are labeled. Glucose carbons are normally composed of the ¹²C carbon isotope. In the measurement of the hepatic recycling constant, (k_{HR}), 1 gram glucose/kg body weight is administered, and the glucose contains an amount of [1, 2-¹³C₂]-glucose. This stable glucose label, [1, 2-¹³C₂]-glucose, has ¹³C carbons at positions 1 and 2 of the 6 carbon chain that forms the backbone of the glucose molecule, and is non-radioactive, hence the term “stable labeled glucose” is used. Although labeling of carbons at positions 1 and 2 are preferred, it will be understood that carbons at other positions may also be used. In one embodiment carbons 1 and 6 are labeled with a non-radioactive isotope. In another embodiment, isotopes of hydrogen bonded to the carbons may also be used as labels as shown in example 2 below.

At block 120, the labeled glucose is administered to a test subject through one of many methods of introducing glucose known in the art such as orally or intravenously. After administration of the stable labeled glucose, passage of the [1, 2-¹³C₂]-glucose into the liver, and exchanges of labeled carbons with the pentose cycle intermediates, to produce glucose molecules having a one ¹³C carbon (termed M1 glucose) instead of two 13C carbons (termed M2 glucose).

At block 130, the disposition of the labeled carbons is measured and evaluated. The fraction of glucose molecules having zero, one or two ¹³C labeled glucose molecules is preferably assessed using gas chromatography/mass spectrometry (GC/MS). The appearance of the newly formed M1 glucose in plasma can only be the result of several specific enzymatic reactions. These include glucose uptake by glucokinase to phosphorylate glucose, oxidation and recycling of the trapped glucose back to glucose via the oxidative and non-oxidative limbs of the pentose cycle, and release of the trapped glucose (glucose 6 phosphate) by glucose-6-phosphatase.

The pentose phosphate (PPP)/glycoytic/gluconeogenic pathway interactions are well known. Generally, control of gluconeogenesis and glycolysis is exerted by modulating the activities of the enzymes which catalyze the three substrate cycles: glucose/glucose-6-P (Glu/Glu-6P), fructose-6-P/fructose-1,6-P2 (Fru-6P/Fru-1,6-P2) and pyruvate/phosphoenolpyruvate (Pyr/PEP). The Glu/Glu-6P, Fru-6P/Fru-1,6-P2 and Pyr/PEP substrates are catalyzed by glucokinase/glucose-6-phosphatase, 6-phosphofructo-1-kinase/fructose-1,6-bisphosphatase and pyruvate kinase/PEPCK, respectively. Additionally, the G6P pool receives flux cycling to and from glycogen, and flux to and from the non-oxidative limb of the pentose phosphate pathway. The non-oxidative PPP flux circulates through the Fru-6P/Fru-1,6-P2 pool, and equilibrates then with the G6P pool, which is the source for oxidative PPP flux. After meals, when both insulin and glucose would be high, flux through G6PDH and TA/TK raises pentose phosphate levels (ribose-5-phosphate (R-5-P), xylulose-5-phosphate (Xu-5-P)). Flux through key gluconeogenic enzymes is inhibited, and the flux through key glycolytic and PPP enzymes is stimulated. It will be understood that successive loss of labeled glucose carbon at C₁-C₂ can occur through the loss catalyzed by glucose-6-phosphate dehydrogenase via the oxidative limb of the pentose cycle producing M1 glucose. ¹³C carbon in the lower half of the glucose molecule that cycles through the non-oxidative limb of the pentose cycle remains intact.

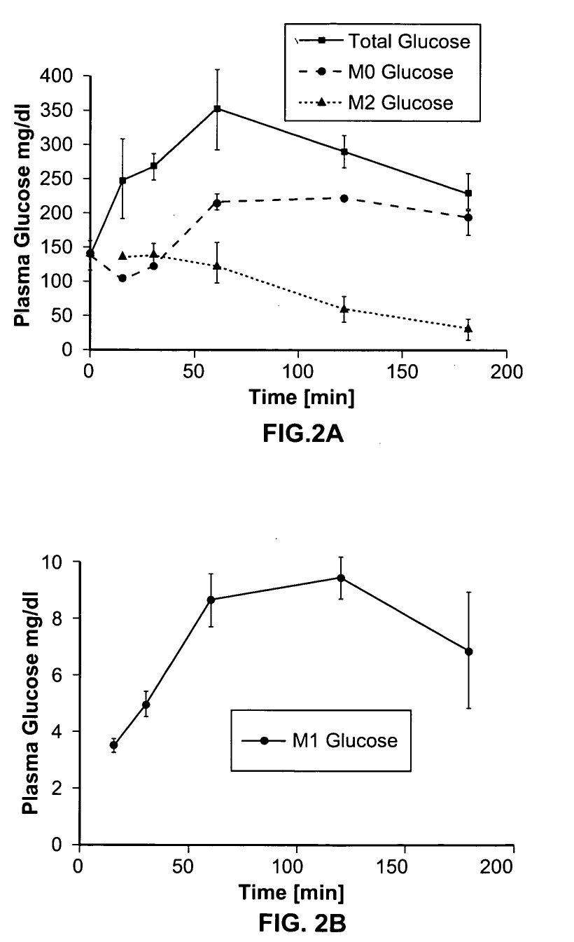

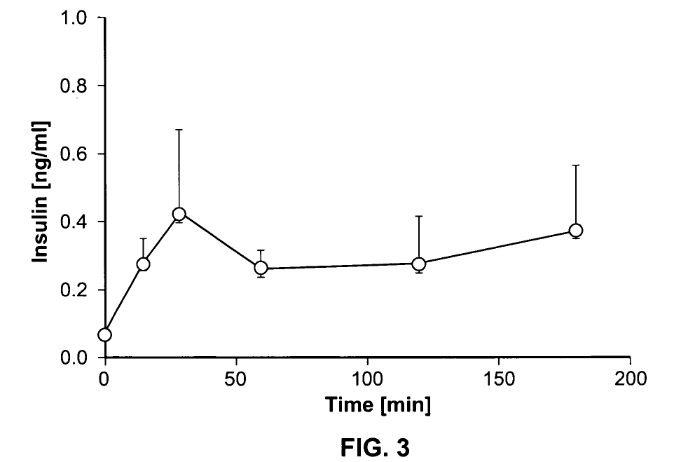

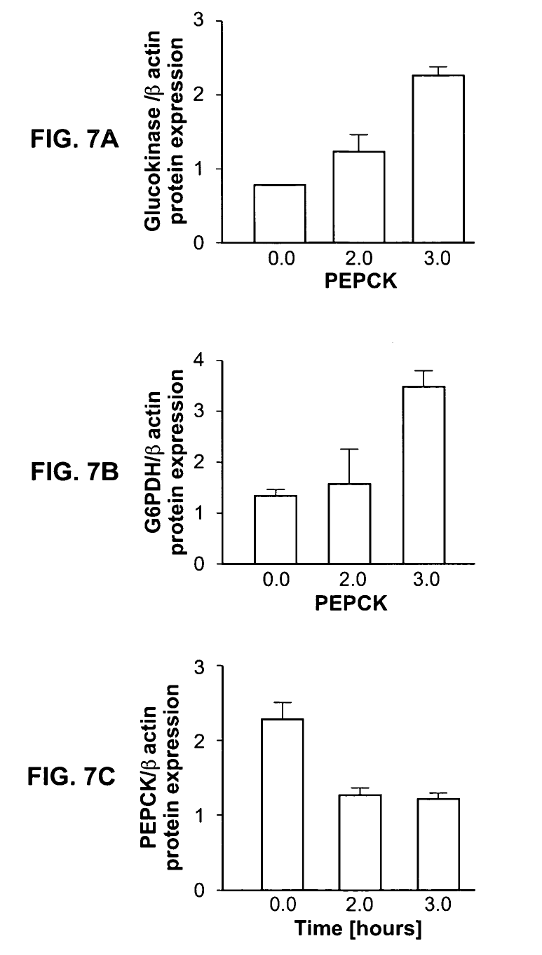

As seen in the examples below, the re-circulation of glucose through the pentose cycle is a process that involves glucokinase and glucose-6-phosphatase, which are both insulin sensitive enzymes. A constant relationship exists in the presence of changing levels of plasma glucose and M2 isotopomer (FIG. 2), insulin concentration (FIG. 3), and changes in the expression of intrahepatic GK, G6PDH and PEPCK protein levels (FIG. 7). It can be seen that excessive hyperglycemia in diabetics during a glucose infusion is due to a decrease in irreversible glucose uptake, and an increase in hepatic futile cycling. Irreversible glucose uptake is the net balance between glucose uptake and glucose production. Impaired phosphorylation in the liver and peripheral tissues leads to a decrease in glucose uptake, while the lack of suppression of glucose production in the liver leads to an increase in glucose recycling.

At block 140, the levels of other enzymes, hormones and other molecules associated with metabolism such as insulin, glucagon or leptin and the like are optionally measured. Such an array of measurements can be compared to baseline levels obtained from healthy populations as well as from populations diagnosed with hyperglycemia or hypoglycemia in block 150 along with the label tracing results. The correlation of measurement results with known recycling constants and baseline levels enable the identification of the locus of certain defects in glucose metabolic pathways and to distinguish between peripheral and hepatic insulin sensitivity, for example.

In the embodiment shown in FIG. 1, the method 100 can be used to screen candidate drugs for use in treating hyperglycemia or hypoglycemia. Alternatively, the method can also be used to determine if a prescribed course of drug treatment is effective in treating a particular patient. At block 150, the administration of labeled glucose and analysis steps are repeated after the test subject is treated with the candidate drug and comparing the results to see if there was any improvement in the condition of the test subject.

In an alternative embodiment, the method 100 can be used in research settings to evaluate genetic mutations in engineered mice, for example, to study the physiological consequences of such mutations. Flux and recycling can be correlated with genetic expression.

The invention may be better understood with reference to the accompanying examples, which are intended for purposes of illustration only and should not be construed as in any sense as limiting the scope of the present invention as defined in the claims appended hereto.

### EXAMPLE 1

To demonstrate the intraperitoneal glucose tolerance test (HRGTT) and glucose recycling, a [1, 2-¹³C₂]-glucose (an M2 glucose isotopomer) load was used in 4-month old C57BL6 mice. Stable isotopes of the M2 isotopomer glucose was administered at 1 mg glucose/gm body weight by intraperitoneal injection. Animals were euthanized by an overdose of isoflurane anesthesia, and tissue from liver and skeletal muscle were rapidly dissected free, snap-frozen in liquid nitrogen, and stored at −80° C. until processed for isolation of RNA or glycogen.

Cytosolic protein was extracted from the liver tissue after homogenization with 10 strikes in lysis buffer containing 0.25 M sucrose, I OmM Tris-HCL(pH7.4), 3 mM MgCL2, 0.1 mM PMSF, 20 mM NaF, 1 mM Na3VO4, 1 MM Na4P207, 1 μg/ml of leupeptin/aprotinin/pepstatin. The resulting cell lysate was filtered with 4 layers of cheesecloth. Nuclei were pelleted by centrifugation at 1000 g for 10 min. Mitochondria were precipitated by centrifugation at 15,000 g for 20 min from the supernatant. The cytosolic fraction was isolated as the supernatant obtained from last ultra-centrifugation at 100,000 g at 4° C. for 1 hr. Protein concentration from cytosol were measured by using an absorbance at 595 run (BCA kit from Pierce). 60 1 μg of protein extracts from cytosol were separated by 10% SDS-PAGE. The membrane blots were incubated with anti-GK at 1:2500 (v/v), anti-G6PDH at 1:2000 (v/v), anti-PEPCK at 1:500 (v/v), and anti-p-actin at 1:2000 (v/v), for either 1 hr in western washing buffer at RT or overnight at 4 C after blocking. The blots were hybridized with secondary antibodies coupled to horseradish peroxidase for 40 to 60 min at RT. Immunodetection was accomplished using enhanced chemiluminescence. Density of each band was determined by scanning the exposed film.

The time course of glucose and lactate isotopomers in plasma, and glucose isotopomers in liver glycogen was determined using gas chromatography/mass spectrometry. The M1 glucose isotopomer, in which ¹³C glucose can occupy the carbon 1 position, was produced via the action of the oxidative limb of the pentose phosphate pathway (PPP) on the administered M2 glucose isotopomer, and its re-entry into the gluconeogenic pathway via the non-oxidative limb of the PPP. The absorption of administered glucose was monitored by the time course of M2 isotopomer, and hepatic glucose recycling by the time course of M1 isotopomer of glucose.

It can be seen that glucose release, due to recirculation through the pentose cycle, is proportional to the mean plasma glucose concentration. The recycling of hepatic glucose traverses several substrate cycles including glucose/glucose-6-phosphate, fructose-6-phosphate/fructose-1,6-P2, phosphoenolpyruvate (PEP)/pyruvate, glycogen recycling via glycogenesis/glycogenolysis, and the recycling of hexose-phosphate via pentose phosphate pathway (PPP).

Turning now to FIG. 1, the time courses of plasma glucose concentration, and the concentrations of M0 and M2 glucose isotopomers after an intraperitoneal injection of 1 mg/gm body weight [1, 2-¹³C₂]-glucose can be seen. The concentration of M1 glucose isotopomers is shown separately with a different scale of the y-axis. The [1, 2-¹³C₂]-glucose entered the plasma glucose pool rapidly achieving an enrichment of 55±1.0%.

The appearance of [1, 2-¹³C₂]-glucose was shown to be accompanied by a doubling of plasma glucose concentration in the first 15-30 minutes. Thus, the initial rise in plasma glucose concentration is mainly due to the absorption of [1, 2-¹³C₂]-glucose. The plasma concentration of M2 glucose leveled off between 30 and 60 minutes, while the plasma glucose concentration continued to rise. The plasma glucose level peaked at 60 minutes to 372 mg/dl. Plasma glucose remained elevated after the first 60 minutes despite a steady decline in M2 enrichment to 20.1±1.3%. The decline in M2 was not accompanied by a parallel decline of MO glucose isotopomer, the unlabeled species derived from glycogenolysis or gluconeogenesis. Thus, it can be concluded that the plasma glucose elevation after the first 60 minutes was mainly sustained by hepatic glucose output.

Referring also to FIG. 2, it can be seen that the rapid increase in plasma glucose between 0 and 30 minutes resulted in a rapid increase in plasma insulin concentration, which peaked at 30 minutes, and then remained constant between 60 and 180 minutes.

During the HRGTT test, [1, 2-¹³C₂]-glucose is oxidized in the liver either via the pentose cycle or the tricarboxylic acid cycle (TCA). When [1, 2-¹³C₂]-glucose is oxidized via the PPP, it can be recycled as singly labeled glucose (M1). The appearance of M1 glucose in plasma is the result of recycling of hepatic glucose. The M1 isotopomer of glucose appeared in the plasma as early as 15 minutes after the intraperitoneal injection with an initial enrichment of 1.4:t 0.094%, and peaked between 2 and 3 hours of the test to about 3.24 t 0.18%. The M1 glucose concentrations during the HRGTT are shown in the graph of FIG. 1B. M I glucose level reached −9 mg/dl between 60 and 120 minutes.

Glycogen synthesis and glycolysis both share the same glucose-6-phosphate intermediate. Thus, ¹³C label from [1, 2-¹³C₂]-glucose appeared in liver glycogen and plasma lactate. Glycogen concentration was seen to be higher at 2 hours than at 3 hours. The M2 isotopomer enrichment in glycogen glucose decreased from 1.9% to 0.8% suggesting a rapid turnover of liver glycogen during HRGTT test. Glycogenolysis has been shown to operate by the first-in-first out principle, which would allow a parallel decrease in M2 isotopomer enrichment of glycogen glucose with that of plasma glucose. The time course of the M2 isotopomer enrichment of plasma glucose was greatly diluted by the unlabeled gluconeogenic flux, which may have been routed, in part, via the unlabeled glucose in glycogen.

It can be shown that glycogen deposited is derived mostly from gluconeogenesis (the indirect pathway). Since [1, 2-¹³C₂]-glucose (M2) was administered, the percent of glycogen synthesis through the direct pathway is taken to be the ratio of the plasma and glycogen M2 glucose isotopomers at a given point in time. It has been observed that the proportion of glycogen made by the indirect vs. direct pathway depends upon many factors, such as the route of administration, the metabolic state of the animal, and the size of the glucose load.

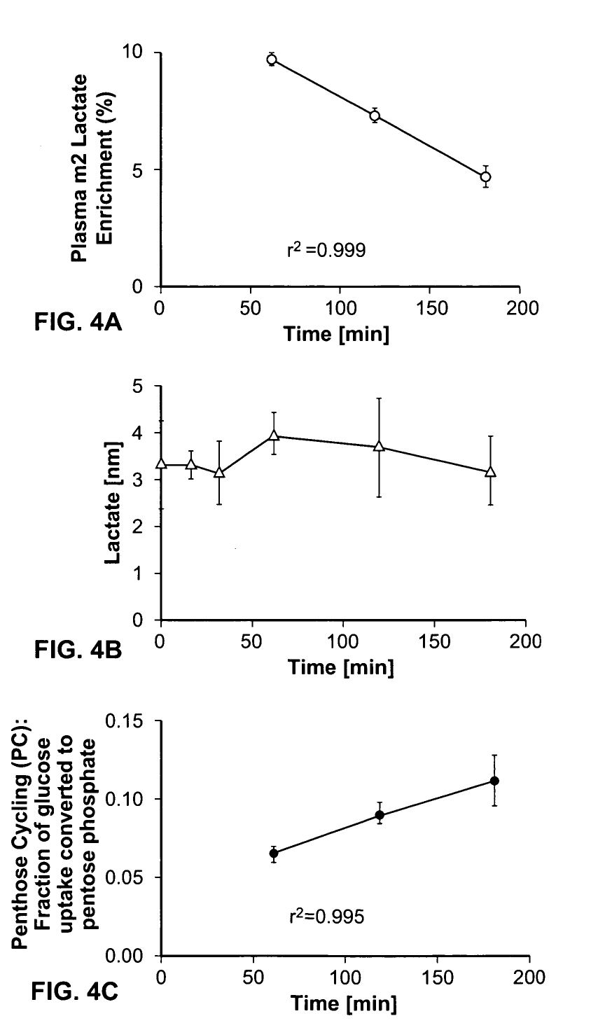

The use of a glucose load that approximates what is given during a glucose tolerance test for humans (1 mg glucose/gin body weight), the direct pathway from glucose uptake contributed less than 5% of the glycogen glucose deposited. In contrast to the reduced M2 glycogen residue enrichment compared to that of plasma glucose (10-fold to 15-fold less at 2 and 3 hours), the MI/M2 ratio of glycosyl residues in glycogen is −3 to 5-fold higher than that in plasma glucose as shown in FIG. 4C.

The observation indicates that the glucose-6-phosphate pool is in equilibrium with intermediates of the pentose and gluconeogenic/glycolytic cycles, but not with the intermediates of glycogenesis/glycogenolysis cycle. The lack of equilibration between the glucose-6-phosphate pool and glycogen is due to the MI/M2 in glycogen being determined by the integration of the history of glucose molecules traveling through the glucose-6-phosphate pool, as retained in glycogen stored, as well as any dynamic recycling occurring via glycogenic/glycogenolytic cycling. Complete equilibration of MI/M2 in glycogen with plasma cannot be expected, as that would imply complete and rapid glycogen turnover, along with glycogen accumulation.

Turning now to FIG. 3A through 3C, the plasma lactate concentration during the HRGTT test can be seen. The plasma lactate concentration was essentially constant throughout the IEPGTT as shown in FIG. 3B. Referring also to FIG. 3A, lactate m2 isotopomer enrichment, which is generated directly as a consequence of the metabolism of [1, 2-¹³C₂]-glucose to triose phosphate, declines from 10% to 4.5% between 60 and 180 minutes and ml lactate enrichment is approximately 2% during the 60 to 180 minutes time period of the HRGTT test. It can also be seen that the rise of m; and m2 lactate lags behind that of the M1 and M2 plasma glucose, suggesting that the isotopomers of lactate are the products of isotopomers of glucose, and that the contribution from pyruvate kinase recycling of lactate via the TCA cycle to m1 lactate is small.

The role of glucose-6-phosphate dehydrogenase (G6PDR) and the non-oxidative branch of the pentose cycle in the formation of M1 glucose can be estimated from the ml/m2 ratio of lactate. In FIG. 3C, the relationship between pentose flux and glycolytic flux indicates a linear increase in the rate of conversion of glucose to pentoses during the same time period in which a linear decrease in the plasma m2 lactate enrichment is seen. These observations indicate an acceleration of pentose cycle flux production during the HRGTT, even as the glucose concentration diminished.

FIG. 3A shows the metabolism of [1, 2-¹³C₂]-glucose to triose phosphate to m2 lactate. The conversion of glucose to lactate is declining with time while pentose cycling (PC), determined as the fraction of glucose uptake converted to pentose phosphate, is increasing linearly with time as shown in FIG. 3C. The recycling of hepatic glucose, leading to the appearance of M1 glucose, occurs when the glucose traverses several substrate cycles including glucose/glucose-6phosphate, fructose-6-phosphate/fructose-1-, 6-P2, phosphoenolpyruvate (PEP)/pyruvate, glycogen recycling via glycogenesis/glycogenolysis, and the recycling of hexose-phosphate via the pentose phosphate pathway (PPP).

The relationship of M2 to M I glucose conversion is shown in FIG. 4A through FIG. 4C. M1 glucose as a fraction of the total labeled glucose pool increases linearly with time is shown in FIG. 4A. Concomitantly, there is a linear decrease with time of M2 glucose as a fraction of the total labeled glucose pool seen in FIG. 4B. In FIG. 4C, glucose M1/M2 ratios are plotted as a function of time and a linear dependence with time is demonstrated. Since M1 is a marker of hepatic glucose output, the change in M1/M2 ratio reflects the rapidity at which M2 glucose is converted to M1 glucose and recycled through hepatic glucose output. M1/M2 glucose increased steadily from 3.6% at 30 minutes to 7.1% at 60 minutes, to 17.6% and 26.1% at 120 and 180 minutes, respectively. However, the total amount of M2 glucose decreased 30% from 60-120 minutes, and decreased 65% from 120-180 minutes as compared to the amount of M2 glucose appearing in plasma between 0-60 minutes seen in FIG. 1. The linear relationship of M1/M2 ratio over time shown in FIG. 4C cannot be predicted from the time courses of the plasma glucose concentration and the plasma-concentrations of its MO, M I and M2 isotopomers seen in FIG. 1. The constancy of the increase in this ratio suggests a constant relationship between hepatic glucose uptake and hepatic glucose output and among the various substrate cycles. Additionally, the flux change is proportional to the concentration of the M2 isotopomer in plasma glucose. In other words, the glucose release, due to re-circulation through the pentose cycle, is proportional to the mean plasma glucose concentration.

Since it is important to know whether hepatic recycling re-circulates through the pentose cycle and/or through the glycolytic and gluconeogenic pathways, the fraction of the ¹³C label present on the upper and lower portions of the M2 glucose isotopomers in plasma glucose, and derived from glycogen was determined. If the [1, 2-¹³C₂]-glucose primarily re-circulates through the oxidative and non-oxidative limbs of the pentose cycle, the M2 glucose isotopomers will primarily be in the upper half of the glucose molecule. If the [1,2-¹³C₂]-glucose re-circulates through the glucose-6-phosphate to triose-phosphate cycle, there will be an appreciable amount of ¹³C label in the lower half of the glucose molecule released by the liver.

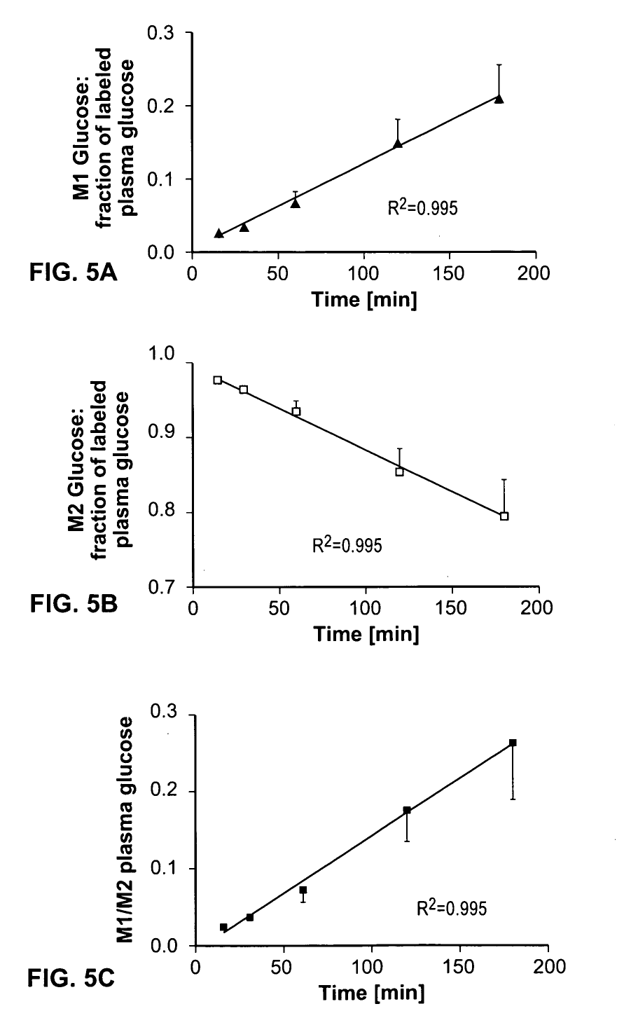

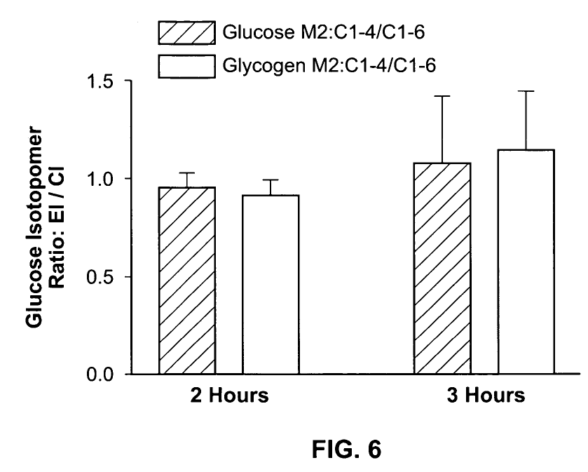

Referring now to FIG. 5, a bar graph of M2 of the C₁-C₄ fragment as a percent of M2 of the C₁-C₆ fragment in both plasma glucose and glucose residues derived from glycogen is shown. For both plasma glucose and glycosyl residues from glycogen, the C₁-C₄ fragment (EI) of the M2 isotopomer accounts for almost all of the M2 isotopomer. This indicates that M2 deposited in glycogen is deposited directly, rather than secondary to re-circulation and recombination of the M1 glucose isotopomer below the triose phosphate level.

The relationship between hepatic glucose recycling and the effect of insulin can be seen by the expression study of glucokinase and glucose-6-phosphate dehydrogenase. The time dependent plot of glucokinase, glucose-6-phosphate dehydrogenase and PEPCK protein expression is shown in FIG. 6. Western Blot analysis showed that hepatic glucokinase (GK) expression rose three-fold and glucose-6phosphate dehydrogenase (G6PDH) expression 2.5-fold. PEPCK expression dropped 50%, relative to P-actin over the 3 hours of the test. These molecular changes are all consistent with the known effect of insulin's regulation of these enzymes, possibly contributing to glucose recycling. The lack of contribution by the direct pathway to glycogen synthesis despite a robust induction of glucokinase and G6PDH expression, and a decrease in PEPCK expression by insulin suggests that the glucose-6-phosphate formed is first routed into the pentose cycle rather than being routed into glycogen synthesis. The redirection of glucose flux into the pentose cycle is probably a function of the degree of elevation of plasma glucose seen during an HRGTT test.

### EXAMPLE 2

In order to evaluate the effect of insulin-responsive gene expressions on substrate fluxes and cycling in the PPARα KO mouse, measurements of the hepatic gluconeogenic flux, glucose absorption, clearance and recycling using the stable isotope isotopomer distribution methods according to the invention.

The expression of insulin dependent gluconeogenic/glycolytic/pentose cycle enzymes was compared to insulin responsiveness for peripheral versus hepatic substrate flux, and futile cycling, in the PPARα KO mouse. The PPARα KO mouse is a model of fasting hypoglycemia due to disordered fatty acid metabolism. It has been previously shown that the hypoglycemia occurred despite an elevated hepatic glucose production, suggesting increased peripheral glucose utilization as the etiology of hypoglycemia in the PPARα KO mouse. However, the elevated glucose production and gluconeogenesis was resistant to the suppression by insulin suggesting hepatic insulin resistance.

Wild-type (C57BL/6) mice and PPARα KO mice were obtained. In this example, glucose was labeled with either stable isotopes of [1, 2-¹³C₂]-glucose, or [2-²H]- and [6, 6-²H₂]-glucose and all isotopes were 99% enriched.

The [1, 2-¹³C₂]-glucose, or a 1:1 mixture of [2-²H]- and [6, 6-²H₂]-glucose, was then administered at 1 mg glucose/gm body weight by intraperitoneal injection. Blood was sampled at 0, 0.5, 1, 2, and 3 hours for [1, 2-¹³C₂]-glucose isotopomer analysis, and 0.5, 1 and 2 hours for deuterated glucose isotopomer analysis.

Plasma glucose and lactate concentrations were determined by the use of a COBAS MIRA analyzer.

All isotopomeric determinations were performed on a Hewlett Packard Mass Selective Detector (model 5973A) connected to an HP Gas Chromatograph (model 6890) using chemical ionization (CI) with 20% methane.

Assessment of glucokinase, glucose-6-phosphatase, pyruvate kinase, pyruvate carboxylase, PEPCK, glucose-6-phosphate dehydrogenase and transaldolase enzyme mRNA levels was accomplished by TAQMAN (Applied Biosystems) RT-PCR.

An insulin tolerance test (ITT), or intraperitoneal glucose tolerance test (HRGTT) were used to investigate the role of PPARα KO in glucose homeostasis in terms of insulin sensitivity or resistance.

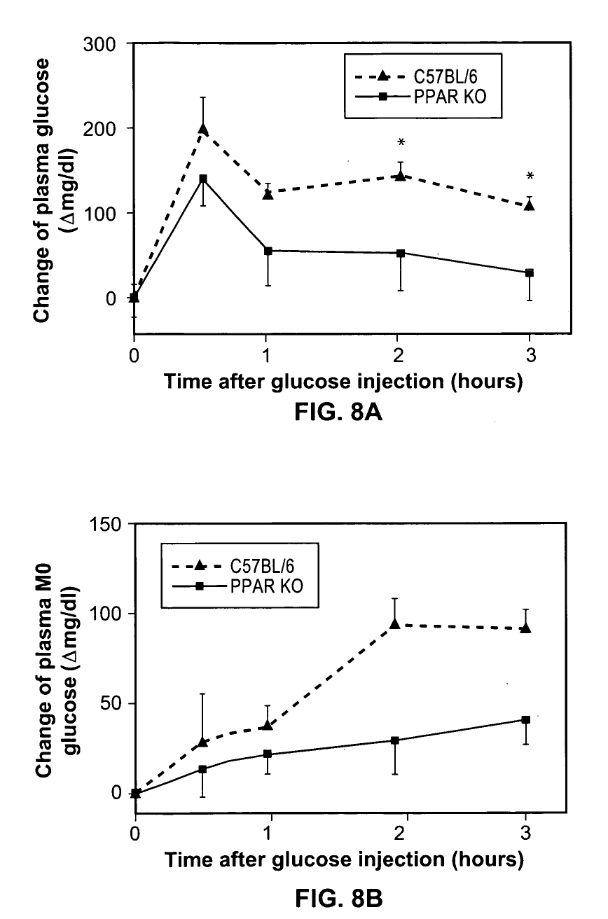

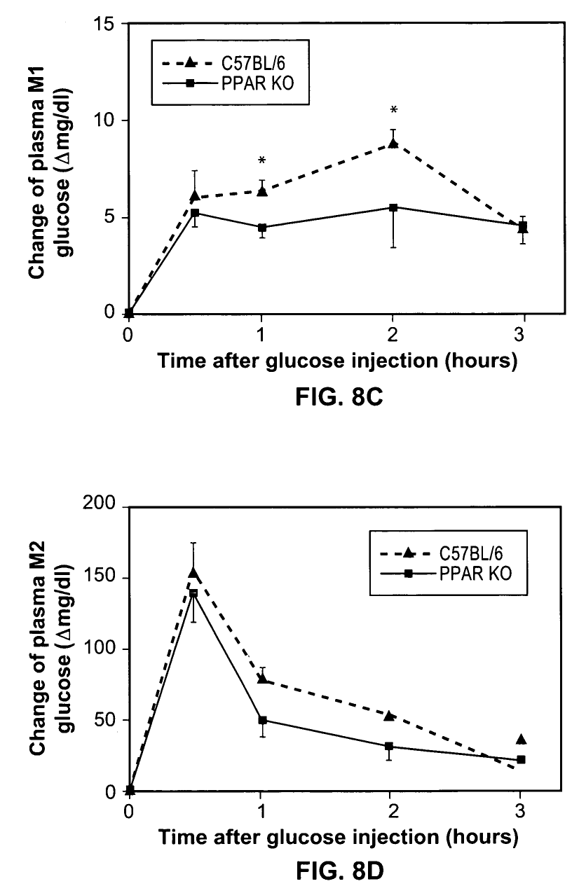

Referring now to FIG. 8A through FIG. 8D, the time course of total plasma glucose and plasma M0, M1 and M2 glucose from basal in response to [1, 2-¹³C₂]-glucose HR-GTT (1 mg/gram body weight) is shown. The time courses of change in total glucose concentration in response to HR-GTT of the PPARα KO and the wild type are shown in FIG. 8A. This figure shows that plasma glucose levels at 0.5, 1, 2, and 3 hours are significantly different from their basal levels in C57BL/6 mice (P<0.01) and in PPARα KO mice (P<0.05) in the two-tailed Student's t-test. M0 glucose (unlabeled glucose) includes glucose produced from unlabeled precursors through gluconeogenic and glycogenolytic pathways, glucose recycled from M2 and M1 glucose isotopomers, as well as glucose recycled from M0 glucose itself (M0 glucose->unlabeled triose->glucose). Thus, plasma M0 glucose levels during the HRGTT reflect the balance between glucose utilization, glucose re-cycling and hepatic glucose production (HGP).

The results shown in FIG. 8B indicate that a significant difference exists in the time course of the level of M0 glucose during the HR-GTT. Following the injection, the increase in plasma M0 glucose for the PPARα KO mice was less than that of the C57BL/6 control.

The M0 time course reflects the integrated response of liver and the periphery to the action of insulin during the HR-GTT. It has been previously shown that HGP and gluconeogenesis are increased as a result of increased glucose-glycerol cycling between liver and adipose tissue, and decreased Cori cycling between liver and muscles is observed. Thus, the lower levels of plasma M0 glucose seen in PPARα KO mice in FIG. 8B were not caused by the increase in HGP and gluconeogenesis, but rather by a decrease in glucose cycling and/or increased glucose utilization.

Turning now to FIGS. 8C and 8D, the appearance of M2 glucose in blood is direct evidence of absorption of administered [1, 2-¹³C₂]-glucose. The levels of plasma M2 glucose during the HR-GTT time course in FIG. 8D depend on the balance between glucose absorption and glucose disposal. Plasma M2 glucose can be recycled via liver back to plasma as M1 glucose, due to the loss of a ¹³C at the first position of the glucose in the reaction catalyzed by glucose-6-phosphate dehydrogenase (G6PDH) of pentose cycle.

Alternatively, the plasma M1 glucose can be produced via the Cori and tricarboxylic acid (TCA) cycles. In this case, the [1, 2-¹³C₂]-glucose is first converted to [1, 2-¹³C₂]-lactate (an m2 lactate isotopomer) through the glycolytic pathway. The m2 lactate generated, via the Cori cycle, is converted to m1 PEP via the TCA cycle and then M1 glucose by the gluoneogenic pathway. Here, the loss of a 13C in the m2 lactate is catalyzed by the exchange reactions of the TCA cycle and PEPCK. Thus, the appearance of plasma M1 glucose is the result of the recycling of plasma M2 glucose, through either the pentose cycle, and/or the Cori cycle mentioned above.

The appearance of M1 glucose during [1,2-¹³C₂]-glucose in plasma during the HR-GTT test shown in FIG. 8C is the consequence of modification of the plasma M2 glucose via the oxidative limb of the pentose cycle (G6PDH) or Cori/TCA cycles. The modified labeled glucose can be recycled back to plasma as M1 glucose via hepatic futile cycling (glucose G6P). The calculated area under the curve of plasma M1 glucose over the 3-hour time course of HR-GTT in C57BL/6 mice was 37% higher than in PPARα KO (p<0.01). At 0.5 hours, the rising level of plasma M1 glucose in PPARα KO mice reached its plateau, while the plasma M1 glucose of C57BL/6 mice continued to rise until the 2-hour time point (FIG. 8C). The generation of plasma M1 glucose for both groups of mice indicates active glucose re-cycling during an HRGTT, with a lower degree of re-cycling when PPARα is absent.

Since glucose uptake does not distinguish between tracer (M2 glucose) and tracee (endogenous M0 glucose), the rate of appearance of intraperitoneal-injected [1, 2-¹³C₂]-glucose (an M2 glucose isotopomer) in blood reflects the rate of glucose absorption. Because M2 is not produced endogenously, the fall in tracer concentration following a bolus dose is entirely due to irreversible loss of the glucose/tracer from the plasma pool. Therefore, the rate of disappearance of M2 glucose from plasma reflects the rate of overall glucose clearance.

For both C57BL/6 and PPARα KO mice, the initial rising of plasma M2 glucose to the same level at 0.5 hours indicated a similar absorption rate of administered M2 glucose. The fall in plasma M2 levels between 0.5 hours and 3.0 hours in FIG. 8D was observed to be 32% faster (p<0.001) in PPARα KO mice than in C57BL/6 mice seen in FIG. 8D indicates a greater rate of overall glucose clearance when PPARα is absent.

The re-cycling of hepatic glucose, leading to the appearance of M1 glucose in blood, occurs when the glucose traverses the glucose<->G-6-P, pentose phosphate pathway, and possibly the TCA cycle. The relationship of M2 to M1 glucose conversion has been shown to be dependent on glucose concentration. Thus, when glucose M1/M2 ratios are plotted as a function of time, a linear dependence with time is revealed. The slope of the increase in this ratio gives the in vivo glucose dependent futile re-cycling rate constant of glucose through G-6-P.

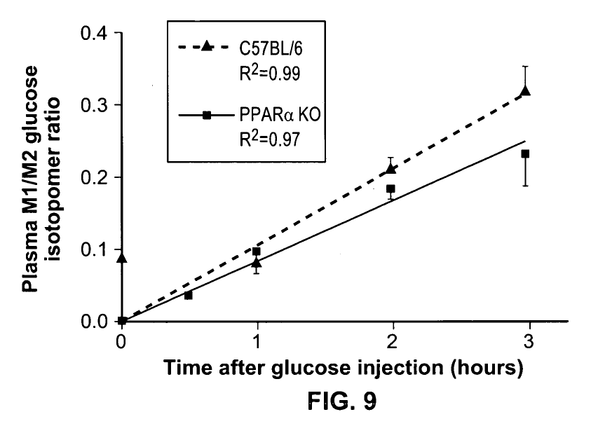

FIG. 9 shows a plot of the ratio of M1 to M2 plasma glucose against time during the [1, 2-¹³C₂]-glucose HR-GTT testing of PPARα KO mice and C57BL/6 mice. The M1/M2 glucose ratio for PPARα KO mice exhibited a time-dependent linearity as expected. The slope of the linear plot gives a glucose recycling rate constant, (k_{HR}). The rate of glucose re-cycling can be expressed as the product of plasma glucose concentration and the re-cycling rate constant, (k_{HR}), and the slope for the line, (k_{HR}), was determined by regression analysis to be 0.1086+0.0049 per hour for C57BL/6, which was significantly higher from the slope of 0.0790+0.0064 per hour for the PPARα KO mice, at p<0.025.

The time-dependent linearity of M1/M2 glucose ratio is believed to be the consequence of two factors: 1) change of plasma M2 enrichment with time; and, 2) the return of a constant fraction of glucose uptake by the liver in futile re-cycling. The change in the M2 glucose enrichment with time is directly dependent on both peripheral and hepatic glucose uptake. The time course of the M1 glucose enrichment is dependent its generation via hepatic re-cycling of plasma M2 glucose taken up by the liver via the pentose cycle, with some contribution from lactate generated from peripheral M2 glucose uptake, via the Cori cycle. Thus, the (k_{HR}) takes on the meaning of the fraction of glucose uptake that is returned through hepatic glucose re-cycling (including the pentose phosphate pathway, and theoretically via the TCA and gluconegenic cycles) per hour. This constant is apparently a physiological property of the liver in response to a glucose challenge.

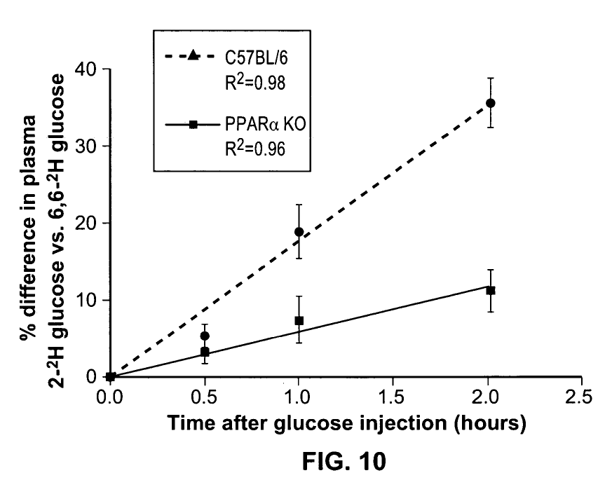

Turning now to FIG. 10, the glucose/glucose-6-P cycling is shown with an alternative label embodiment of the HR-GTT method. The hepatic glucose carbon recycling is the sum process of the TCA cycle, the pentose phosphate cycle and the glucose futile cycle. Hepatic glucose cycling at the level of Gluc/G-6-P is known as glucose futile cycling and is traditionally determined using separate infusions of [2-³H]-glucose and [6-³H]-glucose tracers. The infusion of [2-³H]-glucose is known to provide a different estimate of glucose turnover rate than that from the infusion of [6-³H]-glucose.

The difference is attributed to the fact that tritium in the carbon-2 position of glucose is lost in the equilibrium reaction of isomerization of G-6-P to F-6-P, whereas tritium in the carbon-6 position is retained. Gluc/G-6-P futile cycling equals the difference between glucose turnover as measured by the two tracers.

FIG. 10 shows the results of a modified HR-GTT, using a 1 mg/gm glucose bolus injection composed of equal amounts of the deuterium labeled stable isotopes of [2-²H]-glucose and [6, 6-²H₂]-glucose. Hepatic uptake of [2-²H]-glucose generally leads to the loss of deuterium label at the C2 position due to isomerization between G-6-P and F-6-P.

Hepatic glucose uptake of [6, 6-²H₂]-glucose generally leads to loss of the deuterium label, in part, between the interconversion of pyruvate to lactate, and, in part, between pyruvate and oxaloacetate. When [2-²H]-glucose and [6, 6-²H₂]-glucose are administered as a one to one mixture, the disappearance of the two isotopes, [2-²H]-glucose and [6, 6-²H₂]-glucose can be determined by mass fragmentography by assessing the M1 label in the C1-C4 fragment (for [2-2H]-glucose) and the M2 label in the C3-C6 fragment for [6, 6-²H₂]-glucose) of the EI mass spectrometry. The difference between the two disappearance rates has been recognized as the standard measure of futile cycling (i.e. glucose to glucose-6-phosphate and back).

It can be seen in FIG. 10, that at all times during the [2-²H]/[6, 6-²H₂]-glucose HR-GTT, the % difference between the plasma enrichments of the two tracers is greater for the wild type than for the PPARα KO mouse, indicating a much smaller amount of Gluc/G-6-P futile cycling when PPARα is deficient.

It is apparent that the glucose tolerance testing methods utilizing [1, 2-¹³C₂]-glucose, and the [2-²H]/[6, 6-²H₂]-glucose HR-GTT tests are complementary. It will be seen that the recycling of the original tracer after hepatic modification (to M1 or M0 glucose, or differences in rate of exchange of the [2-²H] versus [6, 6-²H₂] from glucose to water can be quantitatively detected. Glucose absorption and clearance can be compared by following the M2 glucose isotopomer with the use of [1, 2-¹³C₂]-glucose. The peak plasma M2 glucose concentrations were not different between PPARα KO and the wild type C57BL/6 mice, while the clearance of plasma M2 glucose was seen to be slower in the C57BL/6 compared to the PPARα KO mice in FIG. 8. Therefore, injected glucose was absorbed at similar rate but cleared differently in these mice. For the major hepatic glycolytic/gluconeogenic futile cycles, the net flux through Gluc/G-6-P cycle determines the net production or uptake of glucose, and is thus a key in determining blood glucose levels and the tolerance to a glucose load. Increased glucose futile re-cycling has been shown to be associated with overall insulin resistance, mild hyperglycemia and Type 2 diabetes. The background strain, C57BL/6, has a higher rate of hepatic futile cycling, as evidenced in the higher levels of plasma M0 and M1 glucose (FIG. 8) and a higher (k_{HR})(FIG. 9), and a greater relative exchange rate of [2-²H] versus [6, 6-²H₂] from glucose to water (FIG. 10). In the PPARα KO mouse, however, decreased hepatic futile cycling of glucose was observed that compensated for the increased peripheral glucose clearance of the PPARα KO mouse.

The various aspects, modes, embodiments, variations, and features herein shown and/or described are to be considered independently beneficial. However, their various combinations are further contemplated within the intended scope of the invention as would be apparent to one of ordinary skill.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

1. A method for diagnosing diabetes mellitus, comprising:
administering a plurality of labeled glucose molecules to a patient;
quantifying the glucose flux and glucose recycling over time after said administration of labeled glucose; and
comparing quantified glucose flux and recycling with a standard to assess the status of the health of the patient.

2. A method as recited in claim 1, further comprising:
measuring insulin levels at time points after said administration of labeled glucose to said patient; and
correlating said insulin levels with said quantified glucose flux and glucose recycling.

3. A method as recited in claim 1, wherein said labeling comprises:
labeling a first carbon of said glucose at a first end of said glucose molecule; and
labeling a second carbon in said glucose molecule.

4. A method as recited in claim 3, wherein said labeled first carbon is the first carbon to be metabolized during glucose metabolism.

5. A method as recited in claim 1, wherein said label comprises a non-radioactive isotope of carbon.

6. A method as recited in claim 5, wherein said non-radioactive isotope of carbon label comprises a ¹³C isotope.

7. A method as recited in claim 3, wherein said first carbon is labeled with a non-radioactive isotope of carbon and said second carbon is labeled with a non-radioactive isotope of carbon.

8. A method as recited in claim 7, wherein said non-radioactive isotope of carbon label comprises a ¹³C isotope.

9. A method as recited in claim 3, wherein said first carbon is labeled with a non-radioactive isotope of carbon and said second carbon is labeled with a deuterium marker.

10. A method as recited in claim 3, wherein said first carbon is labeled with a deuterium marker and said second carbon is labeled with a deuterium marker.

11. A method for diagnosing diabetes mellitus, comprising:
administering a plurality of labeled glucose molecules to a patient;
quantifying the glucose flux and glucose recycling over time after said administration of labeled glucose;
comparing quantified glucose flux and recycling with a standard to assess the status of the health of the patient;
measuring insulin levels at time points after said administration of labeled glucose to said patient; and
correlating said insulin levels with said quantified glucose flux and glucose recycling.

12. A method as recited in claim 11, wherein said labeling comprises:
labeling a first carbon of said glucose at a first end of said glucose molecule; and
labeling a second carbon in said glucose molecule.

13. A method as recited in claim 12, wherein said labeled first carbon is the first carbon to be metabolized during glucose metabolism.

14. A method as recited in claim 11, wherein said label comprises a non-radioactive isotope of carbon.

15. A method as recited in claim 14, wherein said non-radioactive isotope of carbon label comprises a ¹³C isotope.

16. A method as recited in claim 12, wherein said first carbon is labeled with a non-radioactive isotope of carbon and said second carbon is labeled with a non-radioactive isotope of carbon.

17. A method as recited in claim 16, wherein said non-radioactive isotope of carbon label comprises a ¹³C isotope.

18. A method as recited in claim 12, wherein said first carbon is labeled with a non-radioactive isotope of carbon and said second carbon is labeled with a deuterium marker.

19. A method as recited in claim 12, wherein said first carbon is labeled with a deuterium marker and said second carbon is labeled with a deuterium marker.

20. A method for screening drug candidates for biological activity for potential use in treating a hyperglycemic patient, comprising:
labeling at least one carbon atom of a glucose molecule;
introducing labeled glucose molecules into a mammalian test subject;
introducing a candidate drug into said mammalian test subject;
determining the rate of glucose flux through metabolic pathways in the liver and the peripheral muscles; and
comparing determined flux rates with known baseline flux rates in the absence of said candidate drug.

21. A method as recited in claim 20, further comprising:
measuring hormone and metabolite levels of said test subject; and
comparing said measured hormone and metabolite levels with known baseline levels of said hormone and metabolites in the absence of said candidate drug.

22. A method as recited in claim 20, further comprising:
measuring insulin levels at time points after introduction of labeled glucose into said test subject; and
correlating said insulin levels with said rates of glucose flux in the presence of said candidate drug.

23. A method as recited in claim 22, further comprising:
comparing said measured insulin levels with insulin levels observed in the absence of said candidate drug.

24. A method as recited in claim 20, further comprising:
collecting an array of measurements of flux rates, insulin, hormones and metabolite concentrations from a plurality of healthy individuals;
collecting an array of measurements of flux rates, insulin, hormones and metabolite concentrations from a plurality of individuals with diagnosed hyperglycemia; and
comparing said measurements of flux rates, insulin, hormones and metabolite concentrations from said test subject with said array of measurements from healthy individuals and said array of measurements from individuals diagnosed with hyperglycemia.

25. A method as recited in claim 20, wherein said label of said glucose comprises [1, 2-¹³C₂]-glucose.

26. A method as recited in claim 20, further comprising:
monitoring glucose flux and recycling levels at different concentration levels of candidate drug to determine a minimum effective dose of candidate drug.

27. A method as recited in claim 20, further comprising:
determining the rate of glucose recycling through metabolic pathways in the liver and the peripheral tissues.

28. A method for screening drug candidates for biological activity for potential use in treating a hyperglycemic patient, comprising:
labeling at least one carbon atom of a glucose molecule;
introducing labeled glucose molecules into a mammalian test subject;
introducing a candidate drug into said mammalian test subject;
determining the rate of glucose flux through metabolic pathways in the liver and the peripheral muscles;
comparing determined flux rates with known baseline flux rates in the absence of said candidate drug;
measuring hormone and metabolite levels of said test subject; and
comparing said measured hormone and metabolite levels with known baseline levels of said hormone and metabolites in the absence of said candidate drug.

29. A method for screening drug candidates for biological activity for potential use in treating a hyperglycemic patient, comprising:
labeling at least one carbon atom of a glucose molecule;
introducing labeled glucose molecules into a mammalian test subject;
introducing a candidate drug into said mammalian test subject;
determining the rate of glucose flux through metabolic pathways in the liver and the peripheral muscles;
comparing determined flux rates with known baseline flux rates in the absence of said candidate drug;
measuring insulin levels at time points after introduction of labeled glucose into said test subject; and
correlating said insulin levels with said rates of glucose flux in the presence of said candidate drug.

30. A method as recited in claim 29, further comprising:
comparing said measured insulin levels with insulin levels observed in the absence of said candidate drug.

31. A method for screening drug candidates for biological activity for potential use in treating a hyperglycemic patient, comprising:
labeling at least one carbon atom of a glucose molecule;
introducing labeled glucose molecules into a mammalian test subject;
introducing a candidate drug into said mammalian test subject;
determining the rate of glucose flux through metabolic pathways in the liver and the peripheral muscles;
comparing determined flux rates with known baseline flux rates in the absence of said candidate drug;
collecting an array of measurements of flux rates, insulin, hormones and metabolite concentrations from a plurality of healthy individuals;
collecting an array of measurements of flux rates, insulin, hormones and metabolite concentrations from a plurality of individuals with diagnosed hyperglycemia; and
comparing said measurements of flux rates, insulin, hormones and metabolite concentrations from said test subject with said array of measurements from healthy individuals and said array of measurements from individuals diagnosed with hyperglycemia.

32. A method for screening drug candidates for biological activity for potential use in treating a hyperglycemic patient, comprising:
labeling at least one carbon atom of a glucose molecule;
introducing labeled glucose molecules into a mammalian test subject;
introducing a candidate drug into said mammalian test subject;
determining the rate of glucose flux through metabolic pathways in the liver and the peripheral muscles; and
comparing determined flux rates with known baseline flux rates in the absence of said candidate drug;
wherein said label of said glucose comprises [1, 2-¹³C₂]-glucose.

33. A method for screening drug candidates for biological activity for potential use in treating a hyperglycemic patient, comprising:
labeling at least one carbon atom of a glucose molecule;
introducing labeled glucose molecules into a mammalian test subject;
introducing a candidate drug into said mammalian test subject;
determining the rate of glucose flux through metabolic pathways in the liver and the peripheral muscles;
comparing determined flux rates with known baseline flux rates in the absence of said candidate drug; and
monitoring glucose flux and recycling levels at different concentration levels of candidate drug to determine a minimum effective dose of candidate drug.

34. A method for screening drug candidates for biological activity for potential use in treating a hyperglycemic patient, comprising:
labeling at least one carbon atom of a glucose molecule;
introducing labeled glucose molecules into a mammalian test subject;
introducing a candidate drug into said mammalian test subject;
determining the rate of glucose flux through metabolic pathways in the liver and the peripheral muscles;
comparing determined flux rates with known baseline flux rates in the absence of said candidate drug; and
determining the rate of glucose recycling through metabolic pathways in the liver and the peripheral tissues.
